(19) United States
(12) Patent Application Publication
(10) Pub. No.: US 2021/0378543 A1
(43) Pub. Date: Dec. 9, 2021

(54) ENDOSCOPY SYSTEM AND METHOD OF RECONSTRUCTING THREE-DIMENSIONAL STRUCTURE

(71) Applicant: ALTEK BIOTECHNOLOGY CORPORATION, Hsinchu City (TW)

(72) Inventors: Hsi-Hsin Loo, Taipei City (TW); Te-Wei Yang, Taoyuan City (TW)

(73) Assignee: ALTEK BIOTECHNOLOGY CORPORATION, Hsinchu City (TW)

(21) Appl. No.: 17/409,818

(22) Filed: Aug. 24, 2021

Related U.S. Application Data
(63) Continuation-in-part of application No. 17/023,393, filed on Sep. 17, 2020.

(30) Foreign Application Priority Data
Feb. 13, 2020 (TW) 109201563
Feb. 9, 2021 (TW) 110104881

(51) Int. Cl. A61B 5/06; A61B 1/005; A61B 1/00; H04N 13/239; H04N 13/246; A61B 1/06; G01P 13/00; G01S 17/894

(52) U.S. Cl. CPC A61B 5/067; A61B 1/005; A61B 1/00172; A61B 1/00096; G01S 17/894; H04N 13/246; A61B 1/06; A61B 1/00045; G01P 13/00; H04N 13/239

(57) ABSTRACT

An endoscopy system including a flexible insertion tube, a motion sensing device and a processor is provided. The flexible insertion tube has a central axis. The motion sensing device includes a housing, a plurality of patterns and a plurality of sensors. The patterns are disposed at a surface of the flexible insertion tube according to an axial orientation distribution and an angle distribution based on the central axis. During the relative motion of the flexible insertion tube between the motion sensing device via a guiding hole, the sensors sense a motion state of the patterns so as to obtain a motion-state sensing result. The processor determines an insertion depth information and an insertion tube rotating angle information based on the motion-state sensing result, the axial orientation distribution and the angle distribution. A method of reconstructing a three-dimensional structure is also provided.

### CROSS-REFERENCE TO RELATED APPLICATION

This application is a continuation-in-part application of and claims the priority benefit of U.S. application Ser. No. 17/023,393, filed on Sep. 17, 2020, now pending, which claims the priority benefit of Taiwan application serial no. 109201563, filed on Feb. 13, 2020. This application also claims the priority benefit of Taiwan application serial no. 110104881, filed on Feb. 9, 2021. The entirety of each of the above-mentioned patent applications is hereby incorporated by reference herein and made a part of this specification.

### BACKGROUND

### Technical Field

The disclosure relates to an endoscopy system and a method of reconstructing a three-dimensional structure, and more particularly, to an endoscopy system and a method that can acquire insertion depth information and insertion tube rotating angle information and construct a three-dimensional internal structure of the human body.

### Description of Related Art

An endoscope is an instrument that can be inserted into a human body to diagnose the inside of an organ. Generally, an endoscope is provided with a lens at one end of an insertion tube, and the medical personnel introduce the lens into the human body through the insertion tube to capture an image of the inside of the human body. However, the existing endoscope cannot detect an insertion depth and a rotating angle of the insertion tube. It is difficult for the medical personnel to know an exact location of a lesion. The above information must be obtained with the assistance of other systems. Therefore, when a patient is diagnosed or treated next time, the medical personnel need to spend more time to find lesions found in the previous diagnosis or treatment. It is difficult to achieve accurate medical treatment with the existing endoscope alone, and the diagnostic timeliness is not ideal.

### SUMMARY

The disclosure provides an endoscopy system, which can acquire the insertion depth and rotating angle of the flexible insertion tube and other related information, and can further construct the three-dimensional structure inside the human body, while being able to realize accurate medical treatment and has good timeliness of diagnosis.

In order to achieve the aforementioned objective, the present invention discloses an endoscopy system including a flexible insertion tube, a motion sensing device, an imaging device, and a positioning device. The flexible insertion tube has a central axis. The motion sensing device includes a housing, a plurality of patterns, a plurality of sensors, and a processor. The housing has a guide hole. A plurality of patterns are distributed on the surface of the flexible insertion tube according to an axial orientation distribution based on the central axis. A plurality of sensors are arranged in the housing and adjacent to the guide hole. The processor is arranged in the housing and electrically connected to the sensors. The imaging device is arranged at one end of the flexible insertion tube and connected to the processor. The positioning device is arranged at this end of the flexible insertion tube, and is arranged to obtain the positioning information of this end, and transmit the positioning information to the processor. The flexible insertion tube is inserted into the target body at different depths through the guiding hole. During the relative motion of the flexible insertion tube with respect to the motion sensing device via the guiding hole, these sensors sense the motion state of these patterns to obtain a motion-state sensing result, and the processor determines the insertion depth information according to the motion-state sensing result and the axial orientation distribution. The imaging device generates a plurality of sensing images during the period when the flexible insertion tube is inserted into the target body at different depths. The processor generates a plurality of three-dimensional images by using these sensing images, and combine the three-dimensional images according to the insertion depth information and the positioning information corresponding to the three-dimensional images, so as to reconstruct the three-dimensional structure inside the target body.

In order to achieve the aforementioned objective, the present invention discloses a method of reconstructing a three-dimensional structure comprises: inserting a flexible insertion tube into a target body at different depths through a guiding hole of a motion sensing device; during a relative motion of the flexible insertion tube with respect to the motion sensing device via the guiding hole, sensing a motion state of a plurality of patterns of the motion sensing device by a plurality of sensors of the motion sensing device to obtain a motion-state sensing result; determining an insertion depth information by a processor according to the motion-state sensing result and an axial orientation distribution of the plurality of patterns; generating a plurality of sensing images by an imaging device during the period when the flexible insertion tube is inserted into the target body at different depths; generating, by the processor, a plurality of three-dimensional images by adopting the plurality of sensing images; and combining the plurality of three-dimensional images by the processor according to the insertion depth information and a positioning information at one end of the flexible insertion tube corresponding to the plurality of three-dimensional images to reconstruct the three-dimensional structure inside the target body.

In order to achieve the aforementioned objective, the present invention discloses an endoscopy system comprises a flexible insertion tube, a motion sensing device, an imaging device, a positioning device and a display device. The flexible insertion has a central axis. The motion sensing device comprises a housing and a processor. The housing has a guiding hole, wherein the flexible insertion tube is inserted into a target body at different depths through the guiding hole. the imaging device is disposed at one end of the flexible insertion tube and connected to the processor, wherein the imaging device comprises a light emitting member, an imaging lens, and an image sensor, wherein the light emitting member emits an illuminating beam, the image sensor senses a part of the illumination beam that is reflected from the inside of the target body and penetrates the imaging lens to correspondingly generate a plurality of sensing images. The positioning device is disposed at the end of the flexible insertion tube and obtains a positioning information of the end, and transmit the positioning information to the processor. The display device is connected to the image sensor to display the plurality of sensing images.

Based on the above, in the endoscopy system according to the embodiments of the disclosure, a plurality of patterns of a motion sensing device is disposed at a surface of a flexible insertion tube according to an axial orientation distribution and an angle distribution, and a plurality of sensors is disposed in a housing and adjacent to a guiding hole. Therefore, a distance or angle relationship specified by the patterns is used as a quantitative basis for the description of a location or a motion state. During the relative motion of the flexible insertion tube with respect to the motion sensing device via the guiding hole, the sensors may sense a motion state of the patterns so as to obtain a motion-state sensing result. The processor then determines insertion depth information and insertion tube rotating angle information according to the motion-state sensing result, the axial orientation distribution and the angle distribution. Medical personnel may know the location of a lesion from the insertion depth information and the insertion tube rotating angle information. Therefore, the endoscopy system may achieve accurate medical treatment and has good diagnostic timeliness. In other embodiments of the disclosure, the endoscopy system further includes a positioning device, and uses the processor to combine the insertion depth information, the three-dimensional images, and positioning information to reconstruct the three-dimensional structure inside the human body. Since the three-dimensional images can correspond to different insertion depths, it is possible to avoid dead spots in the three-dimensional images and the reconstructed three-dimensional structure, and the medical accuracy can be greatly improved. In addition, the reconstructed three-dimensional structure can also be stored, providing an important basis for patients in future diagnosis and treatment.

In order to make the foregoing features and advantages of the disclosure more comprehensible, embodiments are described below in detail with the accompanying drawings as follows.

### BRIEF DESCRIPTION OF THE DRAWINGS

FIG. 1A is a schematic application diagram of an endoscopy system applied to a human body according to an embodiment of the disclosure.

FIG. 1B is a schematic appearance diagram of a flexible insertion tube and a motion sensing device of FIG. 1A.

FIG. 1C is a schematic partial cross-sectional diagram of the endoscopy system in FIG. 1A.

FIG. 2A is a schematic enlarged diagram of the flexible insertion tube of FIG. 1A to FIG.

1C during an axial motion and a time-varying diagram of a light intensity electrical signal measured by a corresponding depth sensor.

FIG. 2B is a schematic enlarged diagram of the flexible insertion tube of FIG. 1A to FIG. 1C during a rotating motion and a time-varying diagram of a light intensity electrical signal measured by a corresponding rotating angle sensor.

FIG. 2C is a schematic diagram of a configuration relationship between a plurality of depth sensors and a plurality of patterns and a light intensity electrical signal sensed by a depth sensor.

FIG. 3A is a schematic partial cross-sectional diagram of an endoscopy system according to another embodiment of the disclosure.

FIG. 3B is a schematic enlarged diagram of the flexible insertion tube of FIG. 3A during an axial motion and a time-varying diagram of an electrical signal measured by a corresponding depth sensor.

FIG. 3C is a schematic enlarged diagram of the flexible insertion tube of FIG. 3A during a rotating motion and a time-varying diagram of an electrical signal measured by a corresponding rotating angle sensor.

FIG. 4A is a schematic partial cross-sectional diagram of an endoscopy system according to yet another embodiment of the disclosure.

FIG. 4B is a schematic enlarged diagram of the flexible insertion tube of FIG. 4A during an axial motion and a time-varying diagram of an electrical signal measured by a corresponding depth sensor.

FIG. 4C is a schematic enlarged diagram of a rotating angle sensor corresponding to the flexible insertion tube of FIG. 4A during a rotating motion and a time-varying diagram of an electrical signal measured by a corresponding rotating angle sensor.

FIG. 5 to FIG. 7 are enlarged views of the end portion of the endoscopy system of the embodiment of the disclosure.

FIG. 8 is a block diagram of an endoscopy system according to an embodiment of the disclosure.

### DESCRIPTION OF THE EMBODIMENTS

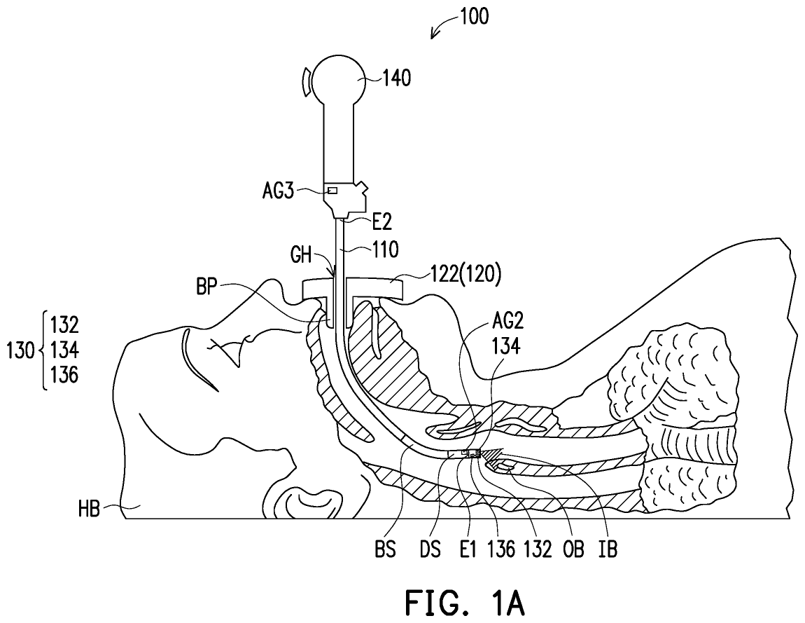

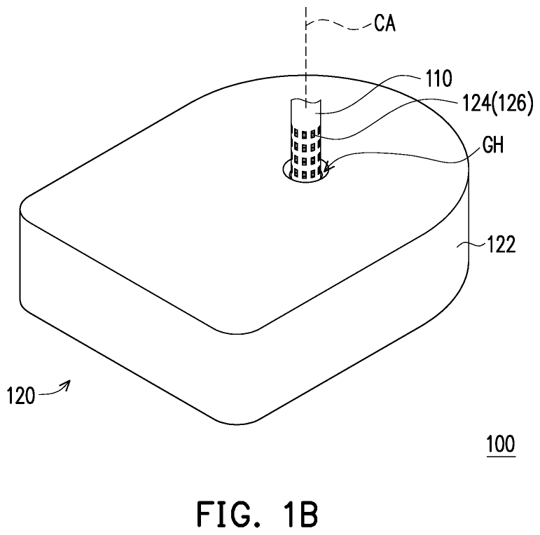

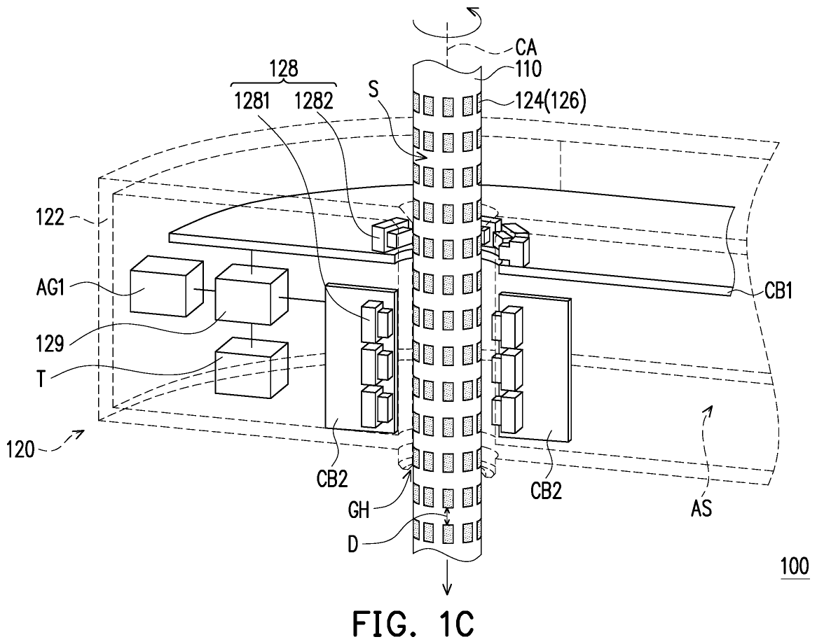

FIG. 1A is a schematic application diagram of an endoscopy system applied to a human body according to an embodiment of the disclosure. FIG. 1B is a schematic appearance diagram of a flexible insertion tube and a motion sensing device of FIG. 1A. FIG. 1C is a schematic partial cross-sectional diagram of the endoscopy system in FIG. 1A.

Referring to FIG. 1A to FIG. 1C, in the present embodiment, the endoscopy system 100 is a medical instrument that enters a human body HB through an insertion tube to observe an internal condition of the human body HB. In detail, the endoscopy system 100 mainly includes a flexible insertion tube 110, a motion sensing device 120, an imaging device 130, and a steering lever 140. In the following paragraphs, a configuration manner among components will be described in detail.

The flexible insertion tube 110 is formed of a flexible material and has flexibility. As shown in FIG. 1B and FIG. 1C, the flexible insertion tube 110 has a central axis CA. An axial orientation referred to in the embodiments of the disclosure refers to an extension direction of the flexible insertion tube 110 along the central axis CA.

The motion sensing device 120 is a device capable of sensing a motion state of the flexible insertion tube 110 by a change in light intensity or a magnetic field. For the convenience of description, the following paragraphs will first take an optical motion sensing device as an example. In the present embodiment, the motion sensing device 120 is, for example, an optical motion sensing device, including a housing 122, a plurality of patterns 124, a plurality of first light emitting members 126, a plurality of sensors 128, a processor 129, a first circuit board CB1, a second circuit board CB2, and a timer T. In the following paragraphs, configurations among internal components of the motion sensing device 120 will be described in detail.

The housing 122 has an accommodating space AS therein for accommodating various components in the motion sensing device 120 and providing a protection function. The housing 122 has a guiding hole GH that communicates with the outside. The flexible insertion tube 110 may enter the human body HB through the guiding hole GH to capture an internal image of the human body HB.

The patterns 124 are disposed at a surface of the flexible insertion tube 110 according to an axial orientation distribution and an angle distribution based on the central axis CA. Specifically, the so-called “disposed at a surface S of the flexible insertion tube 110 according to an axial orientation distribution” means that the patterns 124 are disposed at the surface S of the flexible insertion tube 110 along an axial orientation of the central axis CA according to a specific pitch distribution. The specific pitch distribution is, for example, an equal pitch distribution, that is, in a direction parallel to the axial orientation of the central axis CA, distances D between any two of the patterns 124 are equal to each other, but the disclosure is not limited thereto. In addition, the so-called “disposed at a surface S of the flexible insertion tube 110 according to an angle distribution” means that the patterns 124 are disposed at the surface of the flexible insertion tube 110 by centering on the central axis CA according to a specific angle distribution. The specific angle distribution is, for example, an equal angle distribution, that is, included angles between any two of the patterns 124 relative to the central axis CA are equal to each other, but the disclosure is not limited thereto. The patterns 124 may be optionally disposed on an outer surface or an inner surface of the flexible insertion tube 110, but the disclosure is not limited thereto. Therefore, the patterns 124 have a specified distance or angle relationship as a quantitative basis for the description of a location or a motion state.

The first light emitting members 126 are optical members capable of emitting light functionally, which may be, for example, light emitting components that are electrically controlled to emit light or fluorescent members that are self-luminous without electrical control. The light emitting components are, for example, Light Emitting Diodes (LEDs), Organic Light Emitting Diodes (OLEDs), or other suitable self-luminous electronically-controlled light emitting components. The fluorescent members include fluorescent materials. The disclosure is not limited thereto. A beam emitted by the first light emitting member 126 is referred to as a sensing beam SB. A motion state of the patterns 124 may be sensed by the sensing beam SB. In the present embodiment, the first light emitting members 126 are, for example, integrated into the patterns 124 respectively. Therefore, each pattern 124 may also be regarded as a light emitting pattern.

The sensors 128 senses the motion state of the patterns 124, so as to obtain a motion-state sensing result about the flexible insertion tube 110. In the present embodiment, the sensors 128 are, for example, light sensors capable of converting an optical signal into an electrical signal, which may be, for example, a photodiode. The sensors 128 are disposed in the housing 122 and adjacent to the guiding hole GH. Moreover, according to measuring different motion states, the sensors 128 may further include a plurality of depth sensors 1281 and a plurality of rotating angle sensors 1282. The depth sensors 1281 are disposed along an extension direction of the guiding hole GH and adjacent to the guiding hole GH. The rotating angle sensors 1282 are disposed around the guiding hole GH and adjacent to the guiding hole GH. How to sense the motion state of the patterns 124 will be described in detail in the following paragraphs.

The processor 129 is, for example, an electronic component capable of performing computation, processing or analysis functions on various electrical signals, such as a computer, a Micro Controller Unit (MCU), a Central Processing Unit (CPU), or other microprocessors, Digital Signal Processors (DSP), programmable controllers, Application Specific Integrated Circuits (ASIC), Programmable Logic Devices (PLD) or other similar devices. The processor 129 is disposed in the housing 122 and electrically connected to the sensors 128, so that the processor 129 may receive electrical signals from the sensors 128 to analyze the results.

The first and second circuit boards CB1, CB2 are disposed in the housing 122. The first circuit board CB1 is disposed in the vicinity of an opening of the guiding hole GH, and the guiding hole GH penetrates the first circuit board CB1. The second circuit board CB2 is disposed in the vicinity of a middle portion of the guiding hole GH, and the guiding hole GH penetrates the second circuit board CB2. The first and second circuit boards CB1, CB2 are arranged perpendicular to each other. The depth sensors 1281 are disposed on the first circuit board CB1 and electrically connected to the first circuit board CB1. The rotating angle sensors 1282 are disposed on the second circuit board CB2 and electrically connected to the second circuit board CB2. The processor 129 is electrically connected to the first and second circuit boards CB1, CB2, and receives electrical signals from the depth sensors 1281 and the rotating angle sensors 1282 through the first and second circuit boards CB1, CB2.

The timer T is an electronic component for measuring time, and is electrically connected to the processor 129.

The imaging device 130 is a photoelectric device for capturing an image inside the human body HB, and includes an imaging lens 132, a second light emitting member 134, and an image sensor 136. The imaging device 130 is disposed at an end E1 (for example, tail end) of the flexible insertion tube 110. The imaging lens 132 is, for example, a lens composed of one or more elements with refractive power, which is adapted to receive an image and optically coupled to the image sensor 136. The description of the second light emitting member 134 is similar to that of the first light emitting member 126. The description will be omitted herein. The second light emitting member emits an illumination beam B3 for illuminating an object OB (for example, an organ) to be detected inside the human body HB.

The steering lever 140 is a mechanism member for controlling a motion in the flexible insertion tube 110. The steering lever 140 is disposed at the other end E2 (that is, different from the arrangement end E1 of the imaging device 130) of the flexible insertion tube 110 and coupled to the flexible insertion tube 110. By controlling an angle of a distal segment DS through the steering lever 140, the location of the imaging device 130 adjacent to the distal segment DS may be changed to further detect images of different organs.

In the following paragraphs, the operation mode of the endoscopy system 100 and how to specifically sense the motion-state sensing result of the patterns 124 in the motion sensing device 120 will be explained in detail.

First, the operation mode of the endoscopy system 100 will be described.

Referring to FIG. 1A, a patient may bite a biting portion BP extending below the housing 122 to prevent a user from damaging the flexible insertion tube 110 and fix the motion sensing device 120 above the mouth of the user. The flexible insertion tube 110 may be guided into the human body HB through the guiding hole GH. After the flexible insertion tube 110 enters the human body HB, the second light emitting member 134 emits an illumination beam D3 to illuminate an object OB to be detected (for example, an organ) inside the human body HB. The object OB to be detected reflects at least a part of the illumination beam D3 to the imaging lens 132, and the image sensor 136 senses an image. The image sensor 136 may transmit the image to a back-end display device (not shown) for medical personnel to observe a dynamic image inside the human body HB. In the process of entering the human body HB, medical personnel may directly control an angle of a bending segment BS of the flexible insertion tube 110 through the steering lever 140. Since the distal segment DS of the flexible insertion tube 110 is connected to the bending segment BS, the steering lever 140 may indirectly control the angle of the distal segment DS, and the imaging device 130 may observe different organs in the human body HB as the angle of the distal segment DS changes.

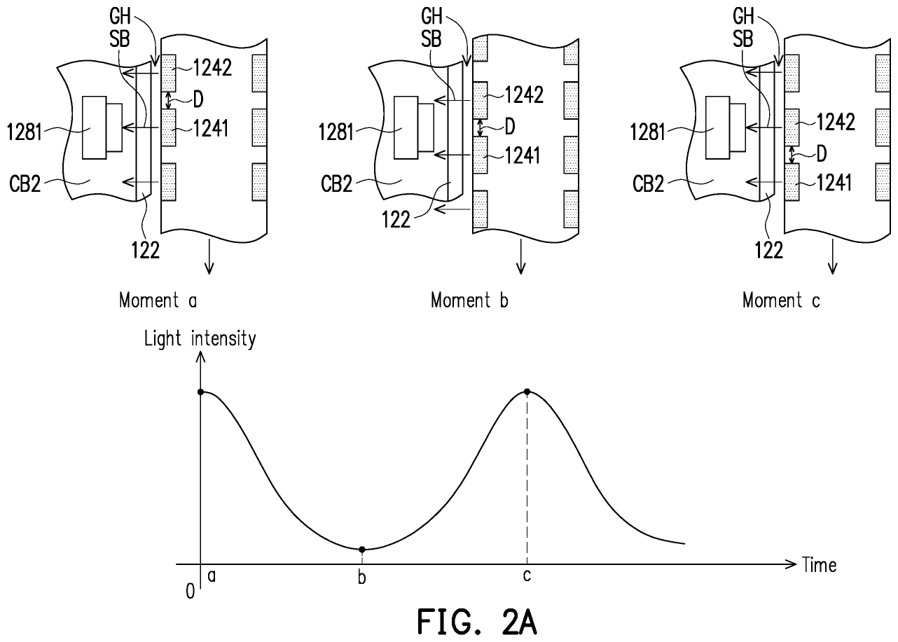

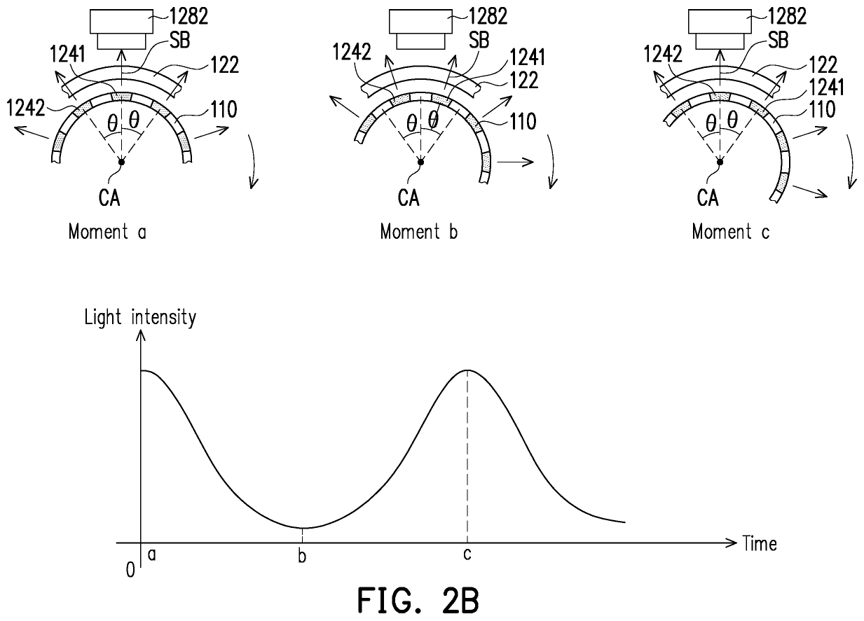

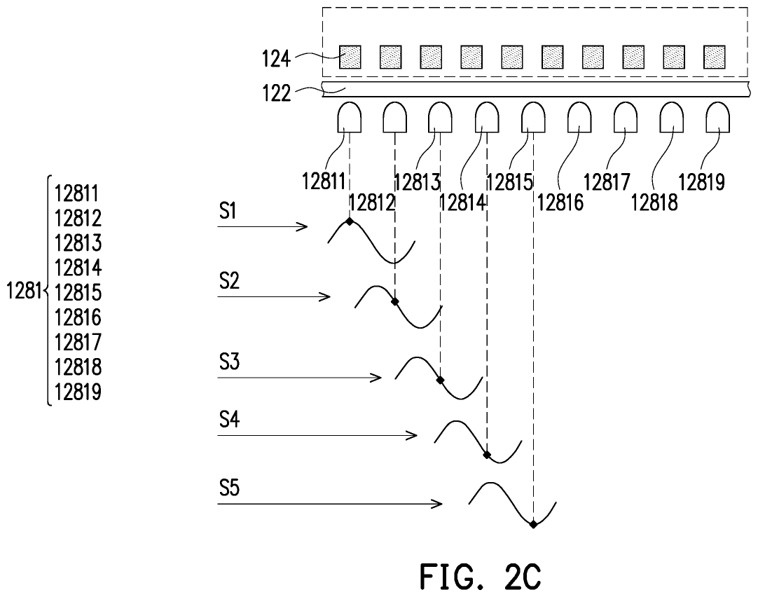

According to the above description, the medical personnel will extend the flexible insertion tube 110 into the human body through the guiding hole GH, and may control the angle of the distal segment by the steering lever 140 to observe different organs inside the human body. The above method results in a relative motion of the flexible insertion tube 110 with respect to the motion sensing device 120. The relative motion includes an axial motion of the flexible insertion tube 110 along the central axis CA and a rotating motion of the flexible insertion tube 110 with respect to the motion sensing device 120. That is, the motion sensing result of the patterns 124 includes an axial motion sensing result and a rotating motion sensing result. In the following paragraphs, FIG. 2A to FIG. 2C are used to explain in sections how the motion sensing device 120 senses an axial motion and a rotating motion.

FIG. 2A is a schematic enlarged diagram of the flexible insertion tube of FIG. 1A to FIG. 1C during an axial motion and a time-varying diagram of a light intensity signal measured by a corresponding depth sensor. FIG. 2B is a schematic enlarged diagram of the flexible insertion tube of FIG. 1A to FIG. 1C during a rotating motion and a time-varying diagram of a light intensity signal measured by a corresponding rotating angle sensor. FIG. 2C is a schematic diagram of a configuration relationship between a plurality of depth sensors and a plurality of patterns and a signal sensed by a depth sensor.

Regarding the mode of sensing an axial motion, a view of a single depth sensor 1281 is taken first. Referring to FIG. 2A, it is assumed that the sensing beams SB emitted by the patterns 124 are integrated into an integrated sensing beam, and it is assumed that the location of the depth sensor 1281 initially corresponds to the center of a pattern 1241 (here labeled 1241, as a light emitting pattern). At this time, the depth sensor 1281 senses a maximum integrated sensing beam light intensity, shown at moment a in FIG. 2A. As the flexible insertion tube 110 travels toward the inside of the human body HB, and assuming that the location of the depth sensor 1281 corresponds to the centers of two patterns 1241, 1242, the depth sensor 1281 senses a minimum integrated sensing beam light intensity, shown at moment b in FIG. 2A. Then, as the flexible insertion tube 110 travels further toward the inside of the human body HB, and assuming that the location of the depth sensor 1281 corresponds to the center of a next pattern 1242, the depth sensor 1281 senses a maximum integrated sensing beam light intensity again, shown at moment c in FIG. 2A. Therefore, for a single depth sensor 1281, as long as the maximum integrated sensing beam light intensity is sensed twice, the size of a distance D by which the flexible insertion tube 110 moves along the axial orientation may be determined. However, other depth sensors 1281 may not be able to sense the maximum integrated sensing beam light intensity twice. Therefore, the back-end processor 129 will perform an operation according to all signal results measured by the depth sensor 1281 to obtain insertion depth information.

Regarding the mode of sensing a rotating motion, referring to FIG. 2B, which is similar to the description of FIG. 2A, it is assumed that the sensing beams SB emitted by the patterns 124 are integrated into an integrated sensing beam, and it is assumed that the location of the rotating angle sensor 1282 initially corresponds to the center of a pattern 1241 (here labeled 1241, as a light emitting pattern). At this time, the rotating angle sensor 1282 senses a maximum integrated sensing beam light intensity, shown at moment a in FIG. 2B. As the flexible insertion tube 110 rotates, for example, the motion sensing device 120 clockwise so that the location of the rotating angle sensor 1282 corresponds to the centers of two patterns 1241, 1242, the rotating angle sensor 1282 senses a minimum integrated sensing beam light intensity, shown at moment b in FIG. 2B. Then, as the flexible insertion tube 110 rotates, for example, the motion sensing device 120 clockwise again so that the location of the rotating angle sensor 1282 corresponds to the center of a pattern 1242, the rotating angle sensor 1282 senses a maximum integrated sensing beam light intensity again, shown at moment c in FIG. 2B. Therefore, for a single rotating angle sensor 1282, as long as the maximum integrated sensing beam light intensity is sensed twice, the size of an angle θ by which the flexible insertion tube 110 rotates clockwise may be determined. However, other rotating angle sensors 1282 may not be able to sense the maximum integrated sensing beam light intensity twice. Therefore, the back-end processor 129 will perform an operation according to all signal results measured by the rotating angle sensor 1282 to obtain insertion tube rotating angle information.

In addition to considering the above factors, the processor 129 will also consider phase factors of the signals measured by the sensors 128 to obtain more accurate insertion depth information and insertion tube rotating angle information. Referring to FIG. 1C, a spatial frequency of the sensors 128 and a spatial frequency of the patterns 122 are different from each other. That is, for the depth sensors 1281, a distance between the two depth sensors 1281 is different from a distance between the two patterns 124 disposed along the axial orientation of the central axis CA. For the rotating angle sensors 1282, an included angle between the two rotating angle sensors 1282 relative to the central axis CA is different from an included angle between the two patterns 124 relative to the central axis CA. Referring to FIG. 2C, a plurality of depth sensors 1281 (for example, but not limited to, 9) and a plurality of patterns 124 (for example, but not limited to, 10) are used as examples for description. It can be seen from this figure that the distance between the two depth sensors 1281 is different from the distance between the two patterns 124. Based on the above configuration, light intensity signal phases measured by each of depth sensors 12811-12819 are more or less different (here only the examples of signals S1-S5 detected by depth sensors 12811-12815 are shown here). Therefore, the processor 129 may further generate a depth coding function for the depth sensors 12811-12819 according to different signal phases, thereby obtaining more accurate insertion depth information. Similar to the method shown in FIG. 2C, the processor 129 may also further generate an angle coding function for the rotating angle sensors 1282 according to different signal phases, thereby obtaining more accurate insertion rotating angle information.

After calculating the insertion depth information and the insertion tube rotating angle information, the processor 129 may integrate the above information to obtain the location of a lesion, and note it in image information for reference by medical personnel. Moreover, the processor 129 may further output the above image and related information to a 3D model manufacturing machine (not shown) for the 3D model manufacturing machine to build an internal model of the human body HB, or as a basis for advanced image processing.

It is to be noted that the above calculation mode is only an example, and in other embodiments, the same parameters (i.e., axial orientation distribution, angle distribution and motion-state sensing result) may also be used to obtain insertion depth information and insertion tube rotating angle information by using different calculation modes. The disclosure is not limited thereto.

Based on the foregoing, in the endoscopy system 100 according to the present embodiment, a plurality of patterns 124 of a motion sensing device 120 is disposed at a surface S of a flexible insertion tube 110 according to an axial orientation distribution and an angle distribution, and a plurality of sensors 128 is disposed in a housing 122 and adjacent to a guiding hole GH. During the relative motion of the flexible insertion tube 110 with respect to the motion sensing device 120 via the guiding hole GH, the sensors 128 may sense a motion state of the patterns 124 so as to obtain a motion-state sensing result. The processor 129 then determines insertion depth information and insertion tube rotating angle information according to the motion-state sensing result, the axial orientation distribution and the angle distribution. Medical personnel may know the location of a lesion from the insertion depth information and the insertion tube rotating angle information. During the next diagnosis and treatment for the patient, the medical personnel may quickly find the lesion according to the previous measurement result, so the endoscopy system 100 may achieve accurate medical treatment.

Further, the processor 129 may further determine speed information and angular speed information of the flexible insertion tube 110 according to time information obtained by the timer T and the insertion depth information and the insertion tube rotating angle information, respectively.

In addition, in the present embodiment, the endoscopy system 100 may further optionally include first to third angle sensors AG1-AG3. In the following paragraphs, the arrangement locations and corresponding functions of the first to third angle sensors AG1-AG3 will be described in detail.

As shown in FIG. 1C, the first angle sensor AG1 is disposed in the housing 122 and electrically connected to the processor 129. The first angle sensor AG1 senses first angle information of the motion sensing device 120 and transmit the first angle information to the processor 129. Therefore, the processor 129 may obtain a horizontal angle, a vertical angle, a tilt angle, or a vibration state of the motion sensing device 120 according to the first angle information, and further calculate the locations of the flexible insertion tube 110 and the lesion. Moreover, the processor 129 may further obtain a change situation of the motion state during the diagnosis and treatment process according to the first angle information and the time information of the timer T.

As shown in FIG. 1A, the second angle sensor AG2 is disposed at an end E1 of the flexible insertion tube 110 and adjacent to the imaging device 130. The second angle sensor AG2 is electrically connected to the processor 129 and senses second angle information of the end E1 of the flexible insertion tube 110. Since the second angle sensor AG2 is closer to the imaging device 130, the second angle information sensed by the second angle sensor may further improve the sensing accuracy of the motion sensing device 120.

As shown in FIG. 1A, the third angle sensor AG3 is disposed on the steering lever 140. The third angle sensor 140 is electrically connected to the processor 129 and senses third angle information of the steering lever 140 to simply sense a rotating angle of the flexible insertion tube 110.

It must be noted here that the following embodiments follow the partial content of the foregoing embodiments, and the description of the same technical content is omitted. For the same component names, reference may be made to the partial content of the foregoing embodiments, and the following embodiments are not repeated.

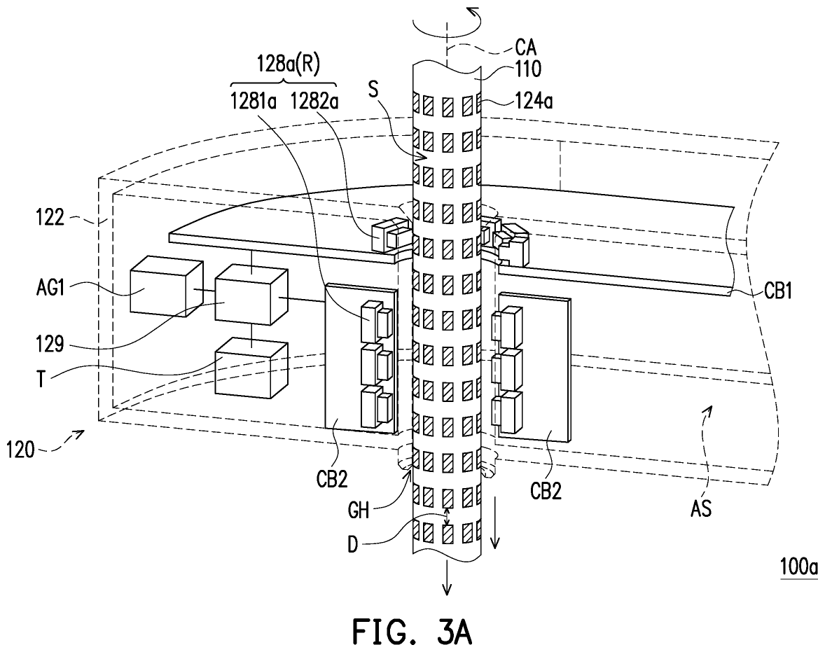

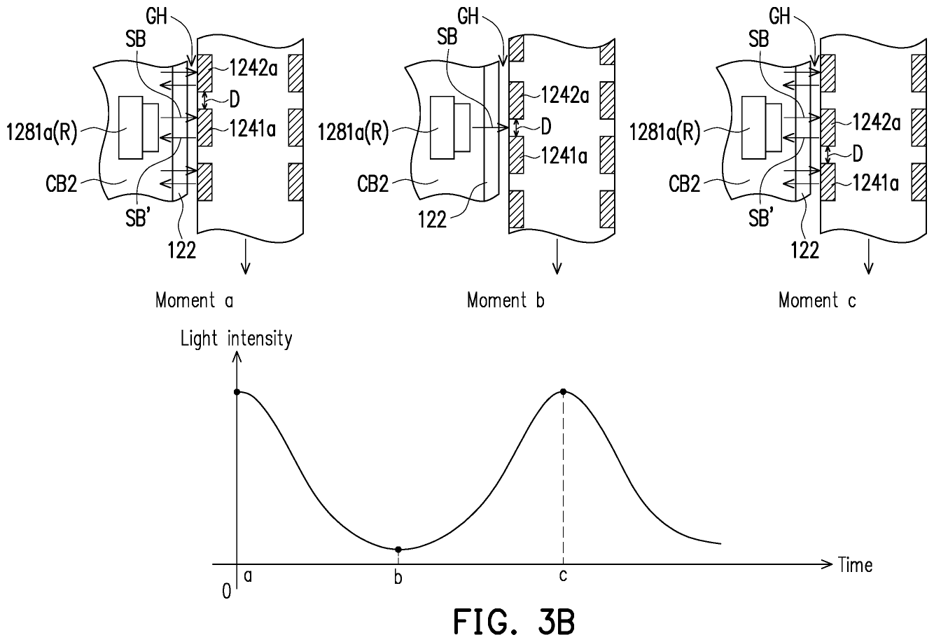

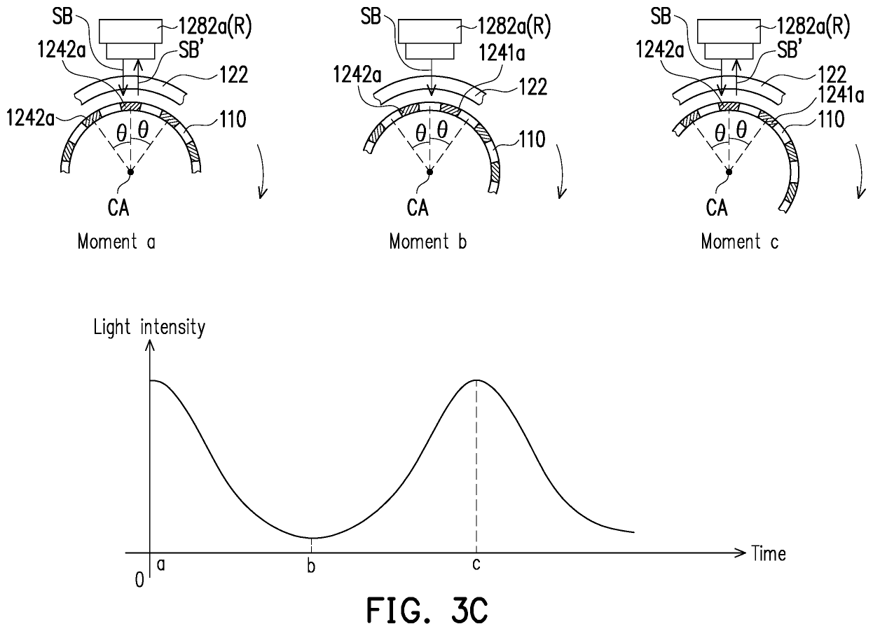

FIG. 3A is a schematic partial cross-sectional diagram of an endoscopy system according to another embodiment of the disclosure. FIG. 3B is a schematic enlarged diagram of the flexible insertion tube of FIG. 3A during an axial motion and a time-varying diagram of an electrical signal measured by a corresponding depth sensor. FIG. 3C is a schematic enlarged diagram of the flexible insertion tube of FIG. 3A during a rotating motion and a time-varying diagram of an electrical signal measured by a corresponding rotating angle sensor.

Referring to FIG. 3A to FIG. 3C, an endoscopy system 100a in FIG. 3A is substantially similar to the endoscopy system 100 in FIG. 1A to FIG. 1C. The main difference is that a motion sensing device 120a in the endoscopy system 100a is a reflective optical motion sensing device. In detail, the patterns are reflection patterns 124a having a reflection function, and the first light emitting elements (not shown in FIG. 3A) are integrated with the sensors 128 (light sensors) respectively. Therefore, each of the first light emitting elements and the corresponding sensor 128 constitute an optical transceiver sending device R.

Referring to FIG. 3B and FIG. 3C, the optical principle of the endoscopy system 100a of the present embodiment is slightly different from the optical principle of the endoscopy system 100. The difference is that during the relative motion of the flexible insertion tube 110 with respect to the motion sensing device 120 via the guiding hole GH, the first light emitting members 126 respectively emit a plurality of sensing beams SB (briefly shown as one) from where the light sensors 128 are located. Sensing beams SB′ reflected by the reflection patterns 124a are transmitted to the depth sensors 1281 and the rotating angle sensors 1282 to obtain an axial motion sensing result and a rotating motion sensing result. The description of the measurement is similar to the related description of FIG. 2A to FIG. 2C and will be omitted herein.

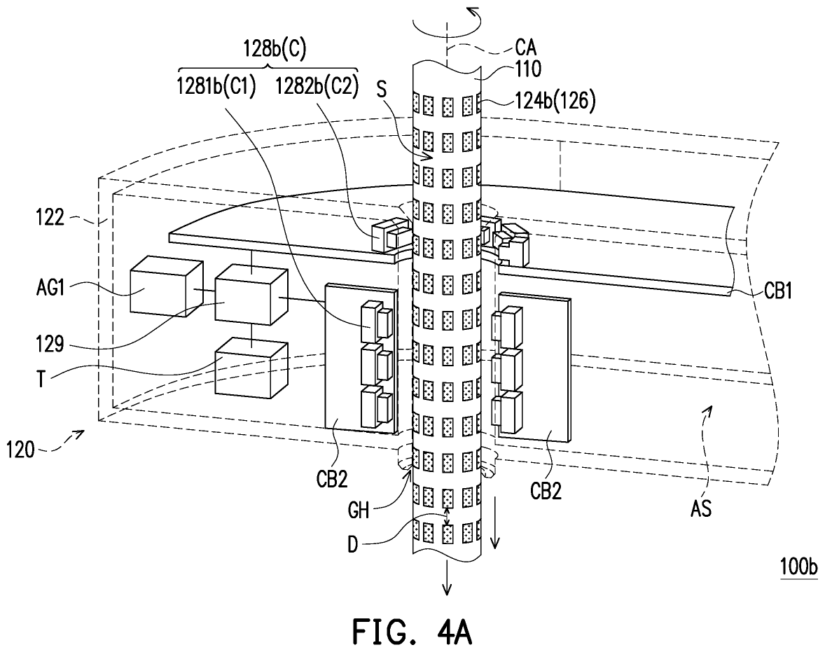

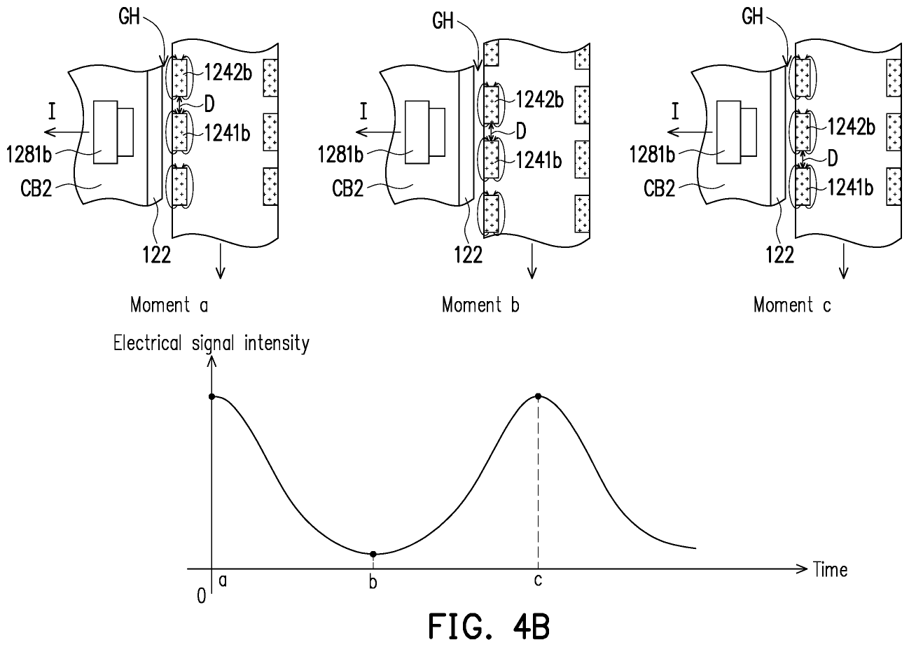

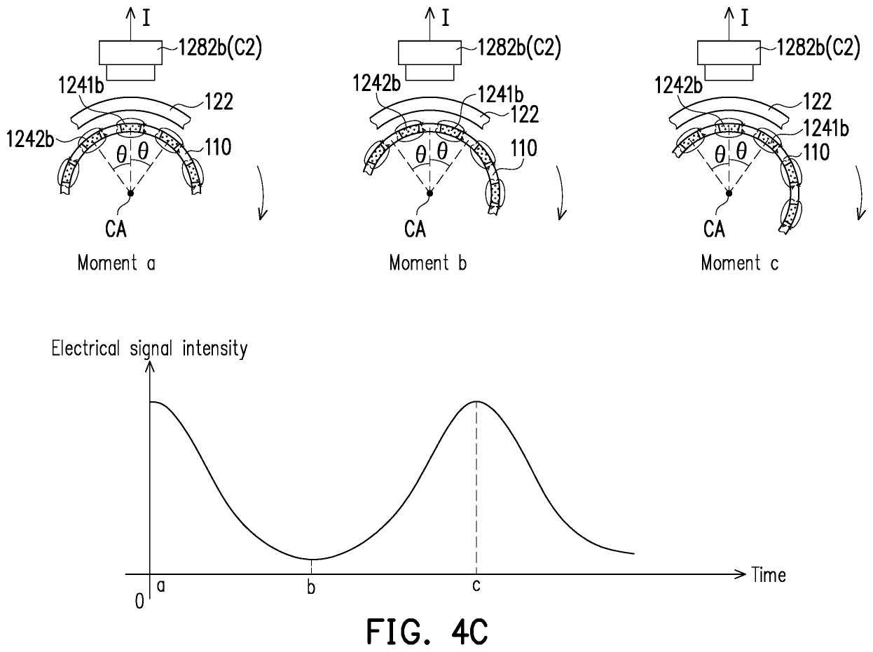

FIG. 4A is a schematic partial cross-sectional diagram of an endoscopy system according to yet another embodiment of the disclosure. FIG. 4B is a schematic enlarged diagram of the flexible insertion tube of FIG. 4A during an axial motion and a time-varying diagram of an electrical signal measured by a corresponding depth sensor. FIG. 4C is a schematic enlarged diagram of a rotating angle sensor corresponding to the flexible insertion tube of FIG. 4A during a rotating motion and a time-varying diagram of an electrical signal measured by a corresponding rotating angle sensor.

An endoscopy system 100b in FIG. 4A is substantially similar to the endoscopy system 100 in FIG. 1A to FIG. 1C. The main difference is that a motion sensing device 120b in the endoscopy system 100b is a magnetic field motion sensing device. In detail, the patterns are a plurality of magnetic patterns 124b, and the sensors 128b are a plurality of induction coils C. That is, the depth sensors 1281b are a plurality of depth induction coils C1, and the rotating angle sensors 1282b are a plurality of rotating angle induction coils C2. For example, the magnetic pattern 124b has, but not limited to, two magnetic lines.

Referring to FIG. 4B and FIG. 4C, the measurement principle of the endoscopy system 100b in the present embodiment is slightly different from the measurement principle of the endoscopy system 100. The difference is that during the relative motion of the flexible insertion tube 110 with respect to the motion sensing device 120 via the guiding hole GH, the depth induction coils 1281b and the rotating angle sensors 1282b produce at least one induced current I due to a magnetic field change of the magnetic patterns 124b caused by the relative motion, and an axial motion sensing result and a rotating motion sensing result are obtained accordingly. In other words, the signal source mode of the motion sensing device 120b is an electrical signal converted by a magnetic field change, and the signal source mode of the motion sensing device 120b is an electrical signal converted by a sensing beam SB. The measurement mode of the motion sensing device 120b is substantially similar to the description of FIG. 2A and FIG. 2B.

The description thereof is omitted herein.

In other embodiments not shown, the sensors 128b in the motion sensing device 120b in FIG. 4A may also be replaced with Hall sensors. That is, the depth sensors 1281b are a plurality of depth Hall sensors, and the rotating angle sensors 1282b are a plurality of rotating angle Hall sensors. Therefore, the depth sensors 1281b and the rotating angle sensors 1282b may sense a magnetic field change of the magnetic patterns 124b to produce at least one induced voltage, and an axial motion sensing result and a rotating motion sensing result are obtained accordingly.

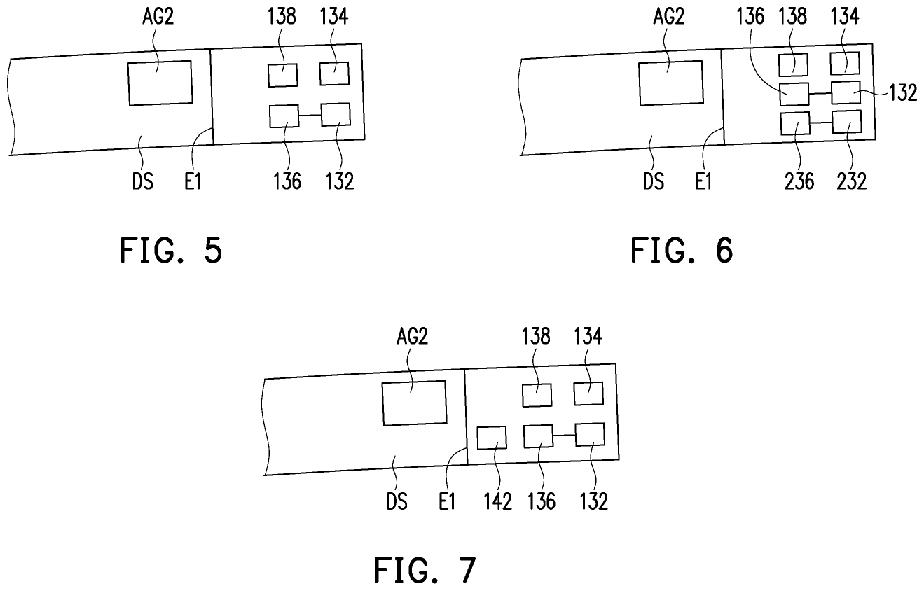

FIG. 5 to FIG. 7 are enlarged views of the end portion of the endoscopy system of the embodiment of the disclosure. The structure of the endoscopy system in these embodiments is substantially the same as that of the endoscopy system 100 shown in FIG. 1A. To avoid repetition, only the structural differences between the endoscopy system in these embodiments and the endoscopy system shown in FIG. 1A are shown.

FIG. 5 is an enlarged view of the end portion of the endoscopy system in embodiment of the disclosure. The structure of the endoscopy system of this embodiment is substantially the same as that of the endoscopy system 100 shown in FIG. 1A, and the differences are described as follows. The endoscopy system of this embodiment further includes a positioning device 138. The positioning device 138 is disposed at one end E1 of the endoscopy system and connected to the processor 129. The positioning device 138 is configured to obtain the positioning information of the end E1 and transmit the positioning information to the processor 129. The imaging device 130 is further connected to the processor 129 and configured to generate a plurality of sensing images during the period when the end E1 is inserted into the human body at different depths.

In an embodiment of the disclosure, the positioning device includes a gyroscope, an accelerometer, and an electronic compass. The gyroscope obtains the angle change information of the end E1 based on the theory of conservation of angular momentum, that is, the orientation angle change of the end E1; the accelerometer senses the acceleration of the end E1, and the integration during the elapsed time can also be adopted to obtain the speed change information and displacement information of the end E1; the electronic compass is configured to sense the orientation information of the end E1, as compared with the property of the gyroscope that obtains the orientation angle change of the end E1, the electronic compass can measure the angle component of the end E1 in the geographic coordinate system. Therefore, in an embodiment of the disclosure, the processor 129 can calibrate the gyroscope according to the electronic compass. In another embodiment of the disclosure, the processor 129 can calibrate the gyroscope based on the insertion tube rotation angle information of the endoscopy system. In addition, in another embodiment of the disclosure, since the insertion depth information of the endoscopy system includes the displacement information of the end E1, the processor 129 can calibrate the positioning information according to the insertion depth information of the endoscopy system.

In the embodiment shown in FIG. 5, the imaging lens 132, the second light emitting member 134 and the image sensor 136 are disposed at the end E1 of the flexible insertion tube, and the second light emitting member 134 can emit the illumination beam as described in the previous embodiment. The image sensor 136 senses the part of the illumination beam reflected from the inside of the human body HB and penetrating the imaging lens 132. The image sensor 136 can correspondingly generate a plurality of first sensing images at different insertion depths. The processor 129 analyzes the first sensing images according to an image processing algorithm (such as a software algorithm) to generate a plurality of corresponding three-dimensional images.

FIG. 6 is an enlarged view of an end portion of an endoscopy system according to an embodiment of the disclosure. The structure of the endoscopy system of this embodiment is substantially the same as that of the endoscopy system shown in FIG. 5, and the differences are described as follows. The imaging device of the endoscopy system of this embodiment further includes an imaging lens 232 and an image sensor 236. The image sensor 236 senses a part of the illumination beam that is emitted from the second light emitting member 134, is reflected from the inside of the human body HB and then penetrates the imaging lens 232, so as to generate a plurality of second sensing images correspondingly. Since the image sensor 136 and the image sensor 236 are disposed on different parts of the end E1, a distance as shown in FIG. 6 is formed between the image sensor 136 and the image sensor 236. The processor 129 can apply the triangulation method to a plurality of first sensing images correspondingly generated by the image sensor 136 and a plurality of second sensing images correspondingly generated by the image sensor 236 to know the distances (i.e., depths) of different tissues, parts or organs in the first sensing images and the second sensing images, thereby generating a plurality of three-dimensional images.

FIG. 7 is an enlarged view of an end portion of an endoscopy system according to an embodiment of the disclosure. The structure of the endoscopy system of this embodiment is substantially the same as that of the endoscopy system shown in FIG. 5, and the differences are described as follows. The endoscopy system of this embodiment further includes a time-of-flight ranging device 142, which is arranged at the end E1 and connected to the processor 129. The processor 129 utilizes the time-of-flight ranging device 142 to perform time-of-flight (ToF) ranging operation within the human body HB, thereby generating three-dimensional depth information. Specifically, in the embodiment shown in FIG. 7, the imaging lens 132, the second light emitting member 134 and the image sensor 136 are disposed at the end E1 of the flexible insertion tube, and the second light emitting member 134 can emit the illumination beam as described in the previous embodiment. The image sensor 136 is configured to sense a part of the illumination beam that is reflected from the inside of the human body HB and penetrates the imaging lens 132, so that the image sensor 136 can correspondingly generate a plurality of first sensing images. The time-of-flight ranging device 142 performs time-of-flight ranging operation within the human body HB corresponding to these first sensing images, so that the processor can obtain the three-dimensional depth information of each first sensing image, and the processor 129 then generates multiple three-dimensional images according to the first sensing images and the corresponding three-dimensional depth information.

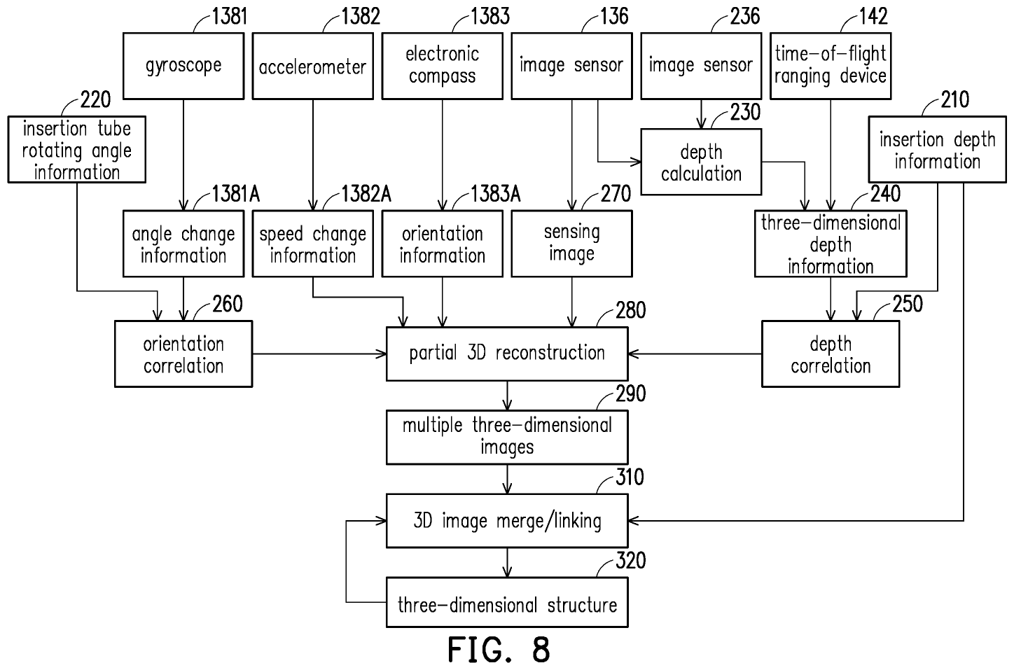

It should be noted that the disclosure provides the embodiments shown in FIG. 5 to FIG. 7 to describe different methods of generating three-dimensional images. The embodiment shown in FIG. 8 to be described below will incorporate some of the technical solutions of FIG. 5 to FIG. 7 and explain how to combine these three-dimensional images according to the insertion depth information, angle change information, and positioning information corresponding to these three-dimensional images to reconstruct the three-dimensional structure inside the human body HB.

It should also be noted that, in the embodiment of the disclosure, the processor 129 is, for example, a central processing unit (CPU), a microprocessor, a digital signal processor (DSP), a programmable controller, a programmable logic device (PLD) or other similar devices or combinations of these devices, the disclosure is not limited thereto. In addition, in an embodiment, the functions of the processor 129 can be implemented as multiple program codes. These program codes are stored in a memory, and the processor 129 executes these program codes.

Alternatively, in an embodiment, each function of the processor 129 may be implemented as one or more circuits. The disclosure provides no limitation to implement the functions of the processor 129 in the form of software or hardware.

Referring to FIG. 8, FIG. 8 shows a block diagram of an endoscopy system according to an embodiment of the disclosure. As mentioned above, the functions of the processor 129 provided by the embodiment of the disclosure can be implemented as multiple program codes. Therefore, in FIG. 8, the various constitution parts of the endoscopy system, including various devices, parts, and functions, are represented by blocks.

The endoscopy system in the embodiment shown in FIG. 8 is provided with insertion depth information 210 and insertion tube rotating angle information 220. The insertion depth information 210 and insertion tube rotating angle information 220 of this embodiment are the same as the insertion depth information and the insertion tube rotating angle information described in the previous embodiment, and thus no further description is incorporated herein. The endoscopy system of this embodiment further includes a gyroscope 1381, an accelerometer 1382, and an electronic compass 1383. The gyroscope 1381 can provide angle change information 1381A, the accelerometer 1382 can provide speed change information 1382A, and the electronic compass 1383 can provide orientation information 1383A.

The endoscopy system of this embodiment further includes an image sensor 136, an image sensor 236, and a time-of-flight ranging device 142, wherein the image sensor 136 can generate a sensing image 270. The image sensor 136 and the image sensor 236 can also respectively generate different sensing images (such as the first sensing image and the second sensing image in the embodiment shown in FIG. 6), and use, for example, the triangulation method to perform depth calculation 230, so as to generate three-dimensional depth information 240. The time-of-flight ranging device 142 can perform time-of-flight (ToF) ranging operation to generate the three-dimensional depth information 240.

Since the insertion tube rotating angle information 220 is associated with the rotating angle of the flexible insertion tube, there is an orientation correlation 260 between the insertion tube rotating angle information 220 and the angle change information 1381A provided by the gyroscope 1381. Similarly, there is a depth correlation 250 between the insertion depth information 210 and the three-dimensional depth information 240.

By combining the orientation correlation 260, the depth correlation 250, the speed change information 1382A, the orientation information 1383A, and the sensing image 270, a partial 3D reconstruction 280 is performed, and a plurality of three-dimensional images 290 can be obtained.

Next, the multiple three-dimensional images 290 are subjected to 3D image merge/linking 310 to reconstruct the three-dimensional structure 320 inside the human body HB. In an embodiment, the processor 129 performs feature comparison on a plurality of three-dimensional images 290 to obtain feature information. Specifically, for example, the feature comparison is performed by looking for and recording the same features in different three-dimensional images, and these information is defined as feature information. The processor 129 acquires the relationship between the different three-dimensional images according to the feature information, and further merges/links these three-dimensional images 290, thereby reconstructing the three-dimensional structure 320 inside the human body HB, and the three-dimensional structure 320 is continuously corrected and compensated in the process to obtain the optimized three-dimensional structure 320.

Based on the foregoing, in the endoscopy system according to the embodiments of the disclosure, a plurality of patterns of a motion sensing device is disposed at a surface of a flexible insertion tube according to an axial orientation distribution and an angle distribution, and a plurality of sensors is disposed in a housing and adjacent to a guiding hole. Therefore, a distance or angle relationship specified by the patterns is used as a quantitative basis for the description of a location or a motion state. During the relative motion of the flexible insertion tube with respect to the motion sensing device via the guiding hole, the sensors may sense a motion state of the patterns so as to obtain a motion-state sensing result. The sensors may sense the motion state of the patterns by optical changes or magnetic field changes of the patterns. Moreover, the sensors further include a plurality of depth sensors and a plurality of rotating angle sensors according to different sensing functions. The depth sensors are disposed in an extension direction of the guiding hole. The rotating angle sensors are disposed around the guiding hole. When the flexible insertion tube undergoes relative motion with respect to the motion sensing device, the depth sensors may sense an axial motion state of the patterns to determine insertion depth information of the flexible insertion tube into a human body. In addition, the rotating angle sensors may sense a rotating motion state of the patterns to determine insertion tube rotating angle information of the flexible insertion tube into the human body. During the next diagnosis and treatment for a patient, medical personnel may quickly find the lesion according to the previous measurement result, so the medical personnel may achieve accurate medical treatment by means of the endoscopy system of the disclosure. In other embodiments of the disclosure, the endoscopy system further includes a positioning device, and uses the processor to combine insertion depth information, three-dimensional images, and positioning information to reconstruct the three-dimensional structure inside the human body, such as the virtual upper or lower digestive tract. Moreover, since the three-dimensional images can correspond to different insertion depths, it is possible to avoid dead spots in the three-dimensional images and the reconstructed three-dimensional structure, which greatly improves the medical accuracy. In addition, the reconstructed three-dimensional structure can also be stored, providing an important basis for patients in future diagnosis and treatment.

Although the disclosure has been disclosed as above by way of embodiments, it is not intended to limit the disclosure. Any person with ordinary knowledge in the technical field can make some changes and decorations without departing from the spirit and scope of the disclosure, so the protection scope of the disclosure shall be determined by the scope of the attached patent application.

What is claimed is:

1. An endoscopy system, comprising:
a flexible insertion tube, having a central axis;
a motion sensing device, comprising:
a housing, having a guiding hole;
a plurality of patterns, disposed at a surface of the flexible insertion tube according to an axial orientation distribution based on the central axis;
a plurality of sensors, disposed in the housing and adjacent to the guiding hole; and
a processor, disposed in the housing and electrically connected to the sensors,
an imaging device, disposed at one end of the flexible insertion tube, and connected to the processor; and
a positioning device, disposed at the end of the flexible insertion tube, and configured to obtain a positioning information of the end, and transmit the positioning information to the processor,
wherein the flexible insertion tube is inserted into a target body at different depths through the guiding hole, during relative motion of the flexible insertion tube with respect to the motion sensing device via the guiding hole, the sensors are configured to sense a motion state of the patterns so as to obtain a motion-state sensing result, and the processor determines an insertion depth information according to the motion-state sensing result and the axial orientation distribution;
the imaging device is configured to generate a plurality of sensing images during the period when the flexible insertion tube is inserted into the target body at different depths, the processor is configured to generate a plurality of three-dimensional images by using the sensing images, and combine the three-dimensional images according to the insertion depth information and the positioning information corresponding to the three-dimensional images to reconstruct a three-dimensional structure inside the target body.

2. The endoscopy system according to claim 1, wherein the processor is configured to perform feature comparison on the three-dimensional images to obtain a feature information, and the processor is configured to combine the three-dimensional images by using the feature information to reconstruct the three-dimensional structure inside the target body.

3. The endoscopy system according to claim 1, wherein the imaging device comprises a light emitting member, a first imaging lens, and a first image sensor, the light emitting member emits an illuminating beam, the first image sensor senses a part of the illumination beam that is reflected from the inside of the target body and penetrates the first imaging lens to correspondingly generate a plurality of first sensing images, the processor is configured to analyze the first sensing images according to an image processing algorithm to generate the three-dimensional images.

4. The endoscopy system according to claim 3, wherein the imaging device further comprises a second imaging lens and a second image sensor, and the second image sensor senses another part of the illumination beam that is reflected from the inside of the target body and penetrates the second imaging lens so as to correspondingly generate a plurality of second sensing images, and the processor is configured to generate the three-dimensional images by using the first sensing images and the second sensing images.

5. The endoscopy system according to claim 4, wherein the processor uses a triangulation method to generate the three-dimensional images.

6. The endoscopy system according to claim 3, further comprising a time-of-flight ranging device, which is arranged at the end of the flexible insertion tube and connected to the processor, and the processor is configured to perform a time-of-flight ranging operation inside the target body by using the time-of-flight ranging device to generate a three-dimensional depth information, and the processor is configured to generate the three-dimensional images according to the first sensing images and the three-dimensional depth information.

7. The endoscopy system according to claim 1, wherein the patterns are arranged at the surface of the flexible insertion tube according to an angle distribution based on the central axis, and the processor is configured to determine an insertion tube rotating angle information according to the motion-state sensing result and the angle distribution.

8. The endoscopy system according to claim 7, wherein the positioning device comprises a gyroscope, an accelerometer and an electronic compass, the processor is configured to calibrate the gyroscope according to the electronic compass or the insertion tube rotating angle information.

9. The endoscopy system according to claim 1, wherein the processor is configured to calibrate the positioning information according to the insertion depth information.

10. A method of reconstructing a three-dimensional structure, comprising:
inserting a flexible insertion tube into a target body at different depths through a guiding hole of a motion sensing device;
sensing, by a plurality of sensors of the motion sensing device, a motion state of a plurality of patterns of the motion sensing device to obtain a motion-state sensing result during a relative motion of the flexible insertion tube with respect to the motion sensing device via the guiding hole;
determining, by a processor, an insertion depth information according to the motion-state sensing result and an axial orientation distribution of the plurality of patterns;
generating, by an imaging device, a plurality of sensing images during the period when the flexible insertion tube is inserted into the target body at different depths;
generating, by the processor, a plurality of three-dimensional images by adopting the plurality of sensing images; and
combining, by the processor, the plurality of three-dimensional images according to the insertion depth information and a positioning information at one end of the flexible insertion tube corresponding to the plurality of three-dimensional images to reconstruct the three-dimensional structure inside the target body.

11. The method of reconstructing the three-dimensional structure according to claim 10, further comprising:
performing, by the processor, a feature comparison on the plurality of three-dimensional images to obtain a feature information; and
combining, by the processor, the plurality of three-dimensional images by using the feature information to reconstruct the three-dimensional structure inside the target body.

12. The method of reconstructing the three-dimensional structure according to claim 10, wherein the imaging device comprises a light emitting member, a first imaging lens, and a first image sensor, the method further comprising:
emitting, by the light emitting member, an illuminating beam;
sensing, by the first image sensor, a part of the illumination beam that is reflected from the inside of the target body and penetrating the first imaging lens to correspondingly generate a plurality of first sensing images; and
analyzing, by the processor, the plurality of first sensing images according to an image processing algorithm to generate the plurality of three-dimensional images.

13. The method of reconstructing the three-dimensional structure according to claim 12, wherein the imaging device further comprises a second imaging lens and a second image sensor, the method further comprising:
sensing, by the second image sensor, another part of the illumination beam that is reflected from the inside of the target body and penetrates the second imaging lens so as to correspondingly generate a plurality of second sensing images, and
generating, by the processor, the plurality of three-dimensional images by using the plurality of first sensing images and the plurality of second sensing images.

14. The method of reconstructing the three-dimensional structure according to claim 13, further comprising:
generating, by the processor, the plurality of three-dimensional images by using a triangulation method.

15. The method of reconstructing the three-dimensional structure according to claim 12, wherein the endoscopy system further comprises a time-of-flight ranging device, which is arranged at the end of the flexible insertion tube and connected to the processor, the method further comprising:
performing, by the processor, a time-of-flight ranging operation inside the target body by using the time-of-flight ranging device to generate a three-dimensional depth information, and generating, by the processor, the plurality of three-dimensional images according to the plurality of first sensing images and the three-dimensional depth information.

16. The method of reconstructing the three-dimensional structure according to claim 10, wherein the plurality of patterns are arranged at a surface of the flexible insertion tube according to an angle distribution based on a central axis of the flexible insertion tube, and the method further comprising:
determining, by the processor, an insertion tube rotating angle information according to the motion-state sensing result and the angle distribution.

17. The method of reconstructing the three-dimensional structure according to claim 16, wherein the positioning device comprises a gyroscope and an electronic compass, the method further comprising:
Calibrating, by the processor, the gyroscope according to the electronic compass.

18. The method of reconstructing the three-dimensional structure according to claim 17, further comprising:
Calibrating, by the processor, the gyroscope according to the insertion tube rotating angle information.

19. The method of reconstructing the three-dimensional structure according to claim 10, further comprising:
Calibrating, by the processor, the positioning information according to the insertion depth information.

20. An endoscopy system, comprising:
a flexible insertion tube, having a central axis;
a motion sensing device, comprising a housing and a processor, and the housing having a guiding hole, wherein the flexible insertion tube is inserted into a target body at different depths through the guiding hole;
an imaging device, disposed at one end of the flexible insertion tube, and connected to the processor, wherein the imaging device comprises a light emitting member, an imaging lens, and an image sensor, wherein the light emitting member is configured to emit an illuminating beam, the image sensor is configured to sense a part of the illumination beam that is reflected from the inside of the target body and penetrates the imaging lens to correspondingly generate a plurality of sensing images;
a positioning device, disposed at the end of the flexible insertion tube, and configured to obtain a positioning information of the end, and transmit the positioning information to the processor; and
a display device, connected to the image sensor and is configured to display the plurality of sensing images.
